(19) United States
(12) Patent Application Publication
(10) Pub. No.: US 2006/0134187 A1
(43) Pub. Date: Jun. 22, 2006

(54) Method for treating inflammation associated with acne

(76) Inventor: Steven Victor, New York, NY (US)

Correspondence Address: CHARU NARANG, ESQ., 101 WEST 55TH STREET, 3C, NEW YORK, NY, 10019, US

(21) Appl. No.: 11/012,911

(22) Filed: Dec. 16, 2004

(51) Int. Cl. A61K 31/573; A61K 9/70

(52) U.S. Cl. 424/449; 514/179

(57) ABSTRACT

The present invention is directed to a method of treating the inflammation associated with acne, comprising applying a patch to an affected area of the skin, wherein said patch includes an efficacious amount of a cortisone. The patch of the present invention may be self adhesive or treated with a water activated adhesive and may be of one layer or multiple layers.

### RELATED APPLICATIONS

This application is related to provisional application No. 60/528,887, filed on Dec. 12, 2003 by Steven Alan Victor, M.D. and claims the filing date accorded to that application.

### FIELD OF INVENTION

The present invention relates to treatment of symptoms of acne. Specifically, the present invention relates to a method for delivering cortisone to an affected area of the skin for the treatment of redness, swelling and inflammation associated with acne.

### BACKGROUND OF THE INVENTION

Acne vulgaris is a disease of the skin that results from the interplay of follicular hyperkeratinization, the presence of Propionibacterium acnes bacteria in the follicular canal and sebum production. It commonly occurs on the face, neck, back and chest, and can vary from quite mild to extremely severe.

Acne usually develops when the sebaceous glands and the lining of the skin duct begin to work overtime, as they do in adolescence. Normally, the lining of the duct sheds cells that are carried to the surface of the skin by the sebum. When the duct is blocked, cells and sebum accumulate, forming a plug (comedo).

If the plug stays below the surface of the skin, it is called a “closed” comedo or whitehead. If the plug enlarges and pops out of the duct, it is called an “open” comedo or blackhead because the top is dark. This is not dirt and will not wash away. The discoloration is due to a buildup of melanin, the dark pigment in the skin.

There are two main types of acne: non-inflammatory and inflammatory. In non-inflammatory acne, there are usually just a few whiteheads and blackheads on the face. A relatively mild type of acne, it can be treated effectively with nonprescription medicines, or as in the case of blackheads, with the prescription drug called Retin-A. The majority of people with acne have this type of acne.

With inflammatory acne, the whiteheads become inflamed, and pimples and pustules develop. In its most severe form, inflammatory acne can cause disfiguring cysts and deep, pitting scars of the face, neck, back, chest and groin. Prescription drugs and sometimes surgery are needed to treat inflammatory acne.

Intralesional cortisone injections are extremely effective in reducing the pain, swelling and redness of acne papules or cysts. Cortisone injection of a diluted corticosteroid into an inflamed pimple, leads to improvement within a day or two, with flattening of the lesion. Cortisone injection therapy is useful as the sole method of treatment of intermittent acne. It is helpful as an additional treatment when acne is not adequately suppressed with antibiotics. This method of treatment involves a doctor or nurse injecting a very small amount of diluted cortisone solution into the pimple or cyst. In 24-48 hours, the pimple goes down significantly or goes away. This type of treatment is painful and can result in a superficial dent if too much is injected or too strong a concentration is injected. The dent usually resolves in many months, yet can be permanent.

An alternative treatment method for reducing the pain, swelling and redness associated with acne is to apply a topical steroid to the skin. However, the drawback to this method is that the ointment will wipe off when it comes into contact with clothing or bed covers, thereby greatly hindering its ability to treat the symptoms it was designed to reduce. One method of preventing the topical ointment from being wiped off is to apply plastic wrap to the affected area and secure it on all sides with tape. However, this method is cumbersome and involves protracted maneuvering of wrapping and tape.

The method of the present invention solves the drawbacks associated with current treatment of the inflammation and redness associated with acne. The patch of the present invention is more efficient in delivery of the cortisone than traditional methods of delivery.

### SUMMARY OF THE INVENTION

The method of the present invention involves treating the inflammation associated with acne, by applying a patch to an affected area of the skin, wherein said patch includes an efficacious amount of cortisone.

The method of the present invention further involves utilizing a patch that is a self adhesive patch.

The method of the present invention involves utilizing a patch that is comprised of a material that is capable of absorbing moisture generated by the skin.

The method of the present invention involves, in the alternative, utilizing a patch that is comprised of a material that is non-porous.

The method of the present invention may also involve a patch that is comprised of at least one layer of material.

The method of the present invention may involve, in the alternative, a patch that is comprised of at least two layers.

The method of the present invention may involve a patch in which the at least two layers are not the same material.

The method of the present invention may involve, in the alternative, a patch in which the at least two layers are the same material.

The method of the present invention may utilize a patch wherein the efficacious amount of cortisone is delivered via a carrier.

The method of the present invention may utilize 0.01 to 5% concentration of cortisone.

### DETAILED DESCRIPTION OF PREFERRED EMBODIMENTS

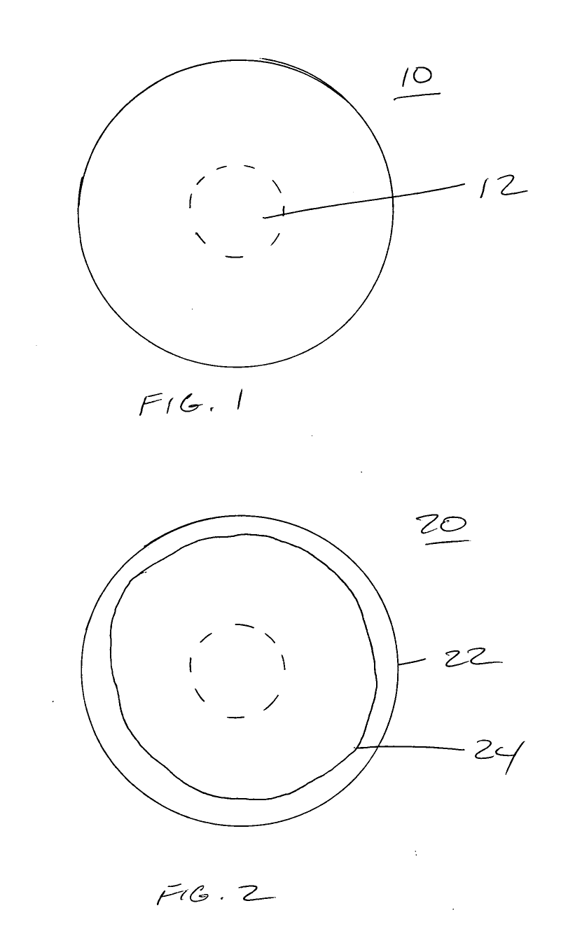

FIG. 1 depicts one preferred embodiment of the patch 10 of the present invention. Although the patch 10 is shown to be circular in form, it may be any shape desired, rectangular or asymmetrical, for example. The patch 10 of the present invention may be made, in one preferred embodiment, of a single layer of a non-porous material, in order to increase absorption of the cortisone into the area to be treated. The patch 10 may be self adhesive or be coated with an adhesive that is activated by water in order to allow it to adhere to the skin. Any suitable commercially available adhesive may be used for this purpose.

The patch 10 may be manufactured such that the cortisone is contained on the patch and the user may simply place the cortisone loaded patch on the area to be treated. The broken line circle 12 illustrates a location on the patch 10 where the cortisone to be delivered to an inflamed area may be contained. However, the cortisone may be placed on the periphery or any other location on the patch as desired or necessary. The cortisone to be delivered to an affected area may be placed on the patch alone or in combination with carrier agents, gels, or any other bio-agent that will not interfere with the use and efficacy of the cortisone. In the alternative, the cortisone may be applied to the skin and the patch may be used to cover the thus treated area, providing a covering and/or moisture seal.

FIG. 2 depicts another preferred embodiment of the patch of the present invention. In this embodiment, the patch 20 is made of two layers of material. The outer layer 22 is a non-porous material as designed to prevent the treated area from coming into contact with extraneous moisture or dirt. The inner layer 24 is a porous material designed to absorb excess moisture from the treated area. Again, the cortisone may be placed on the skin directly and then covered by the patch or the patch may be pre-loaded with cortisone. In this embodiment, the cortisone may be placed on the outer layer 22 and the covered by the inner layer 24. In the alternative, the cortisone may be placed on the inner layer 24. The cortisone may be delivered by any method that does not interfere with its efficacy, such as via a gel or ointment formulation. The patch 20 may be self adhesive or treated with an adhesive that is activated by water.

What is claimed is:

1. A method of treating the inflammation associated with acne, comprising applying a patch to an affected area of the skin, wherein said patch includes an efficacious amount of a cortisone.

2. The method according to claim 1, wherein said patch is a self adhesive patch.

3. The method according to claim 1, wherein said patch is comprised of a material that is capable of absorbing moisture generated by the skin.

4. The method according to claim 1, wherein said patch is comprised of a material that is non-porous.

5. The method according to claim 1, wherein said patch is comprised of at least one layer of material.

6. The method according to claim 1, wherein said patch is comprised of at least two layers.

7. The method according to claim 6, wherein said at least two layers are not the same material.

8. The method according to claim 5, wherein said at least two layers are the same material.

9. The method according to claim 1, wherein said efficacious amount of cortisone is delivered via a carrier.

10. The method according to claim 1, wherein 0.01 to 5% concentration of cortisone is used in the patch.
